(19) United States
(12) Patent Application Publication
(10) Pub. No.: US 2026/0224198 A1
(43) Pub. Date: Aug. 6, 2026

(54) Ultrasound Scanner Mount

(71) Applicant: FUJIFILM SONOSITE, INC., Bothell, WA (US)

(72) Inventors: Ben Dekock, Bothell, WA (US); Craig Chamberlain, Seattle, WA (US); Spencer Denton, North Bend, WA (US); Tyler Dawson, Bothell, WA (US); Kenji Kimura, Bothell, WA (US); Scotty Paton, Bothell, WA (US); Jonathan Schreven, Bellevue, WA (US); Bridget Kocer, Bothell, WA (US)

(21) Appl. No.: 19/046,352

(22) Filed: Feb. 5, 2025

(51) Int. Cl. A61B 8/00

(52) U.S. Cl. CPC A61B 8/4427; A61B 8/4438; A61B 8/462; A61B 2560/0214; A61B 2560/0456

(57) ABSTRACT

Ultrasound systems, scanner mounts, and methods for using the same are disclosed. In some embodiments, an ultrasound system includes an ultrasound scanner configured to transmit ultrasound at a patient anatomy and receive reflections of the ultrasound from the patient anatomy and a display device configured to display an ultrasound image generated by the ultrasound system based on the reflections of ultrasound. The ultrasound system also includes a mount configured to mechanically support the display device, a strut including a first end portion and a second end portion, where the first end portion is configured to couple the strut to the mount, the second end portion is configured to receive a holder, and the holder is configured to hold the ultrasound scanner.

### FIELD

Embodiments disclosed herein relate to ultrasound systems. More specifically, embodiments disclosed herein are related to a mount for a scanner of an ultrasound system.

### BACKGROUND

Ultrasound systems can have scanner mounts configured to hold a scanner. The mounts may obscure writing on the scanner that can indicate a make, model, and frequency range of the scanner, requiring extra time on behalf of the user to select an appropriate scanner for a procedure. The mounts can obscure a battery status indicator of the scanner, so that a user may select a scanner which has insufficient battery power to perform an examination. The mount may be suitable for only scanners manufactured by a same manufacturer of the scanner mount, preventing the charging of scanners manufactured by different manufacturers while the scanner is retained by the mount.

The scanner mount may permit the scanner to be incorrectly oriented in the scanner mount. In some cases, the scanner can fall through the scanner mount and hit the floor, potentially damaging the scanner. In some cases, a user may not be able to visually tell if a scanner is properly mounted. Hence, the user may think the scanner is being charged when it is not being charged, preventing the scanner from being ready for use. Furthermore, the scanner mounts may lack access for cleaning, or be difficult to clean, which can spread germs and contagions.

In some cases, a scanner mount may be made of a material that prevents or inhibits the transfer of charging energy via a non-contact charger.

### SUMMARY

Ultrasound systems, scanner mounts, and methods for using the same are disclosed. In some embodiments, an ultrasound system includes an ultrasound scanner configured to transmit ultrasound at a patient anatomy and receive reflections of the ultrasound from the patient anatomy and a display device configured to display an ultrasound image generated by the ultrasound system based on the reflections of ultrasound. The ultrasound system also includes a mount configured to mechanically support the display device, a strut including a first end portion and a second end portion, where the first end portion is configured to couple the strut to the mount, the second end portion is configured to receive a holder, and the holder is configured to hold the ultrasound scanner.

In some other embodiments, an ultrasound system includes an ultrasound scanner, which includes a light bar, and a scanner mount including an end portion having a band configured to at least partially surround a perimeter of the ultrasound scanner, where the end portion is configured to receive a holder, and the band and the holder are configured to retain the ultrasound scanner, the ultrasound scanner when retained configured to at least partially expose the light bar.

In some embodiments, a scanner mount for holding an ultrasound scanner, includes a band configured to at least partially surround a perimeter of the ultrasound scanner, a backing plate coupled to the band and configured to receive a holder, where the holder is configured to be removably attached to the backing plate, and the band and the holder are configured to retain the ultrasound scanner in the scanner mount. The scanner mount also includes a charger transmitter positioned between the backing plate and the holder and configured to transfer a charging energy through the holder to the ultrasound scanner when the ultrasound scanner is retained by the band and the holder.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound system in an environment using an ultrasound scanner mount during an ultrasound examination.

FIG. 2 illustrates some embodiments of the ultrasound system from FIG. 1.

FIGS. 3A and 3B illustrates some embodiments of an ultrasound system including an ultrasound scanner mount attached to a display device mount.

FIGS. 4A and 4B illustrate some embodiments of an ultrasound scanner mount attached to display device mount.

FIGS. 5A-5C illustrate some embodiments of an ultrasound scanner.

FIGS. 6A-6C illustrate some embodiments of light bars of an example ultrasound scanner.

FIGS. 7A-7B illustrate some embodiments of an ultrasound scanner mount.

FIGS. 8A and 8B illustrates some embodiments of an ultrasound scanner mount.

FIG. 9 illustrates some embodiments of a system.

FIG. 10 illustrates some embodiments of an example device.

FIG. 11 illustrates some embodiments of an example environment.

FIG. 12 illustrates some embodiments of an example machine-learning architecture used to train a machine-learned model.

FIG. 13 illustrates some embodiments of an example machine-learned model using a CNN.

FIG. 14 illustrates some embodiments of an example method.

FIG. 15 illustrates some other embodiments of an example method.

FIG. 16 illustrates some other embodiments of an example method.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Systems, devices, and methods are disclosed herein an ultrasound system scanner mount that holds an ultrasound scanner. In some embodiments, the mount includes a holder to hold the ultrasound scanner and strut to couple the mount to couple and/or secure the mount to a portion of the ultrasound system. In some embodiments, the mount includes a band that at least partially surrounds a perimeter of the ultrasound scanner to retain the ultrasound scanner in the mount. The scanner can include a light bar or other indicia that is at least partially visible when the scanner is in the mount. In some embodiments, a scanner mount includes a charger transmitter to charge the ultrasound scanner when the ultrasound scanner is in the holder.

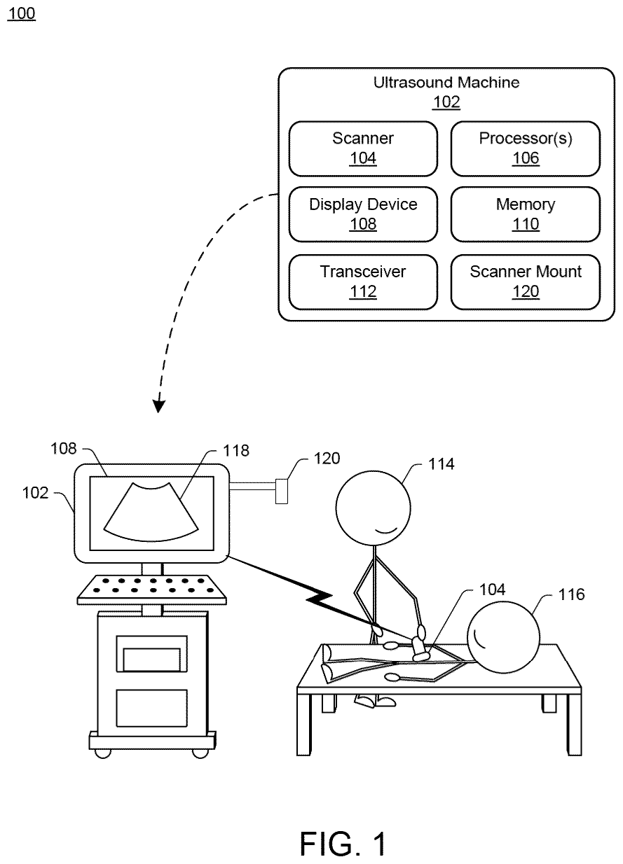

In some embodiments, the ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument. The ultrasound machine 102 includes various components, some of which include the scanner 104, one or more processors 106, a display device 108, a memory 110, a transceiver 112, and a scanner mount 120.

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116 for testing, diagnostic, therapeutic, or procedural reasons. In some embodiments, the scanner 104 includes an ultrasound transducer array and electronics communicatively coupled to the ultrasound transducer array to transmit ultrasound signals to the patient's anatomy and receive ultrasound signals reflected from the patient's anatomy. In some embodiments, the scanner 104 is an ultrasound scanner, which can also be referred to as an ultrasound probe or transducer. In some embodiments, the scanner 104 is a multi-array scanner. For instance, a multi-array scanner in accordance with the present invention can include one or more of the arrays described in U.S. patent application Ser. No. 18/613,694 filed on Mar. 22, 2024, entitled Multi-Dimensional and Multi-Frequency Ultrasound Transducers to Zhang et al. In some embodiments, the multi-array scanner includes one or more of the arrays described in U.S. patent application Ser. No. 17/561,313 filed on Dec. 23, 2021 entitled Array Architecture and Interconnection for Transducers to Li et al.

The display device 108 is coupled to the processor 106, which can include any suitable processor, number of processors, or processor system, such as one or more central processing units (CPUs), graphics processing units (GPUs), vector processors, reduced instruction set computing (RISC) processors, complex instruction set computing (CISC) processors, a very long instruction word (VLIW) processors, etc. The processor 106 can execute instructions stored on memory 110 to perform operations disclosed herein. For example, the processor 106 can process the reflected ultrasound signals to generate ultrasound data, including an ultrasound image. The display device 108 is configured to generate and display an ultrasound image (e.g., ultrasound image 118) of the anatomy and/or interventional instrument based on the ultrasound data generated by the processor 106 from the reflected ultrasound signals detected by the scanner 104. In some embodiments, the ultrasound data includes the ultrasound image 118 or data representing the ultrasound image 118. The transceiver 112 can be configured to transmit, e.g., over a network maintained by a care facility, the ultrasound data and/or any data related to the ultrasound examination, such as medical worksheet data, to a medical archiver (e.g., a vendor neutral archive (VNA)). In embodiments, the transceiver 112 can receive data from the medical archiver, such as patient history data including previous examination data.

In some embodiments, the display device 108 is attached to a display device mount, e.g., a plate or fixture to hold the display device 108. A scanner mount 120 can be attached to the display device mount that supports the display device. The scanner mount 120 can be configured to hold the scanner 104, as described in more detail with respect to the subsequent figures.

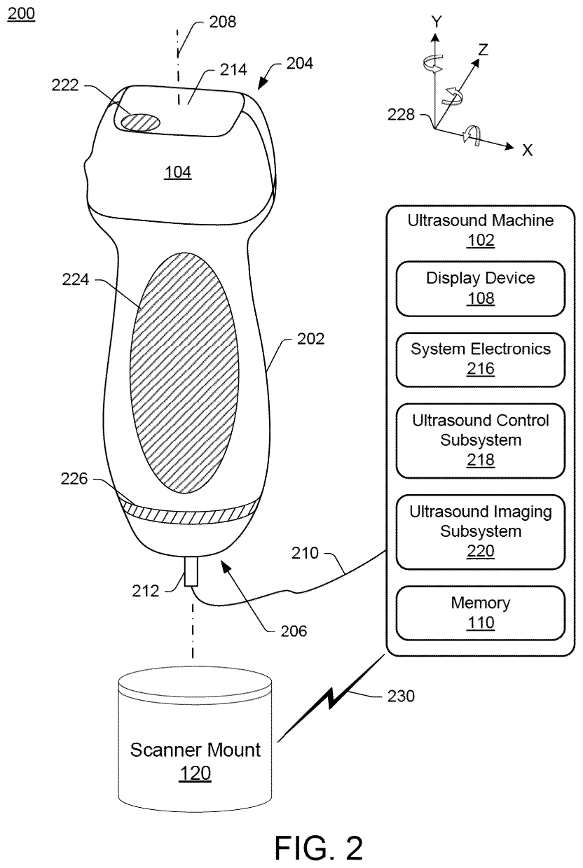

FIG. 2 illustrates some embodiments of the ultrasound system illustrated in the environment 100 of FIG. 1. Referring to FIG. 2, the ultrasound system 200 includes the scanner 104. In some embodiments, the scanner 104 (e.g., ultrasound scanner) can be any suitable type of ultrasound scanner. The scanner 104 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. The scanner 104 is electrically coupled to an ultrasound imaging system (e.g., the ultrasound machine 102) via a coupling 210. In some embodiments, the coupling 210 includes a cable that is attached to the proximal end portion 206 of the scanner 104 by a strain-relief element 212. In some embodiments, the coupling 210 includes a wireless coupling so that the scanner 104 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth. In some embodiments, the ultrasound scanner includes a low-frequency array and a high-frequency array which can work in conjunction to extend the overall bandwidth of the ultrasound scanner. For instance, the system can implement harmonic imaging which transmits low-frequency ultrasound via a first array and receives echoes including harmonics of the ultrasound on a second array.

In some embodiments, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some embodiments, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receives electrical pulses from the scanner 104 that were created from the returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some embodiments, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In some embodiments, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

In some embodiments, the ultrasound scanner 104 also includes one or more pressure sensors 222 on the lens of the scanner 104-1, and one or more pressure sensors 224 on the enclosure 202 of the scanner 104. The pressure sensors 222 and 224 can include in, on, or under a sensor region any suitable type of sensors for determining a pressure. In some embodiments, the pressure sensors 222 and 224 includes capacitive sensors that can measure a capacitance, or change in capacitance, caused by a user's touch or proximity of touch, as is common in touchscreen technologies. The pressure sensors 222 and 224 can generate sensor data indicative of a touch or pressure. The sensor data can include a binary indicator that indicates the presence and absence of a touch on the sensor. For instance, a “1” for sensor data can indicate that a pressure is sensed at the pressure sensor, and a “0” for the sensor data can indicate that a pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity, and generally can be of any suitable shape and size and generate sensor data indicating pressure at any suitable number of points. For instance, in some embodiments, the pressure sensors 222 cover an exterior surface of the lens of the scanner 104 and can used to determine when the scanner is placed against a patient. Additionally or alternatively, the pressure sensors 224 can substantially cover the enclosure 202 of the scanner 104 and can be used to determine when a clinician grabs the scanner 104 for use in an ultrasound examination (e.g., the clinician has a suitable grip on the scanner 104 to perform the ultrasound examination), or when the scanner 104 is placed in a scanner mount. In some embodiments, the scanner mount includes pressure sensors similar to the pressure sensors 222 and 224 on the scanner 104, which can be implemented to determine a position of the scanner in the scanner mount.

In some embodiments, the scanner 104 includes an inertial measurement unit (IMU) 226 for generating positional data that determines a position and orientation of the scanner 104 in a coordinate system, e.g., the coordinate system 228 in FIG. 2. The IMU can include a combination of accelerometers, gyroscopes, and magnetometers, and generate positional data including data representing six degrees of freedom (6DOF), such as yaw, pitch, and roll angles in the coordinate system. Typically, 6DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera and fiducial markers on the scanner 104 (not shown in FIG. 2) to determine the positional data for the ultrasound scanner 104.

In some embodiments, the system generates, based on the positional data from the IMU 226 and/or sensor data from the pressure sensors 222 and 224, a trigger signal. For instance, the system can determine a position of the scanner 104 in the scanner mount 120 and generate a trigger signal based on the position indicting a correct positioning and orientation in the scanner mount 120. The trigger signal can be used to initiate charging of a battery in the scanner 104 from a non-contact charger included in the scanner mount. In some embodiments, the system uses the positional data from the IMU 226 and/or sensor data from the pressure sensors 222 and 224 to make a determination of a manufacturer of the ultrasound scanner 104. For instance, the system can implement a machine-learned model that processes the sensor data indicative of contact with the scanner mount to make the determination. In some embodiments, the sensor data includes pressure data from sensors on the scanner mount, as well as the pressure sensors 222 and 224 on the scanner 104. The determination can include whether the scanner 104 and the scanner mount 120 are manufactured by a same manufacturer. The system can adjust the charging of the scanner 104 based on the determination, including to reduce or disable charging energy from the scanner mount 120 to the scanner 104 when the scanner 104 is manufactured by a different manufacturer than the manufacturer of the scanner mount 120. In some embodiments, the scanner includes multiple arrays, and the system enables one or more of the arrays based on the trigger signal, so that the scanner 104 is immediately enabled for use once it is lifted from the scanner mount 120.

In some embodiments, implementation 200 also includes the scanner mount 120. In some embodiments, the scanner mount 120 is implemented to mount and hold the scanner 104. For example, the scanner 104 can be inserted along the central axis 208 into the scanner mount 120. The scanner mount 120 can be communicatively coupled to the ultrasound machine 102, e.g., via the coupling 230 that can include a wireless communication link and/or a cable. The scanner mount 120 can include a charging system to transfer charge to the scanner 104 when it is inserted into the scanner mount 120, as described below in more detail.

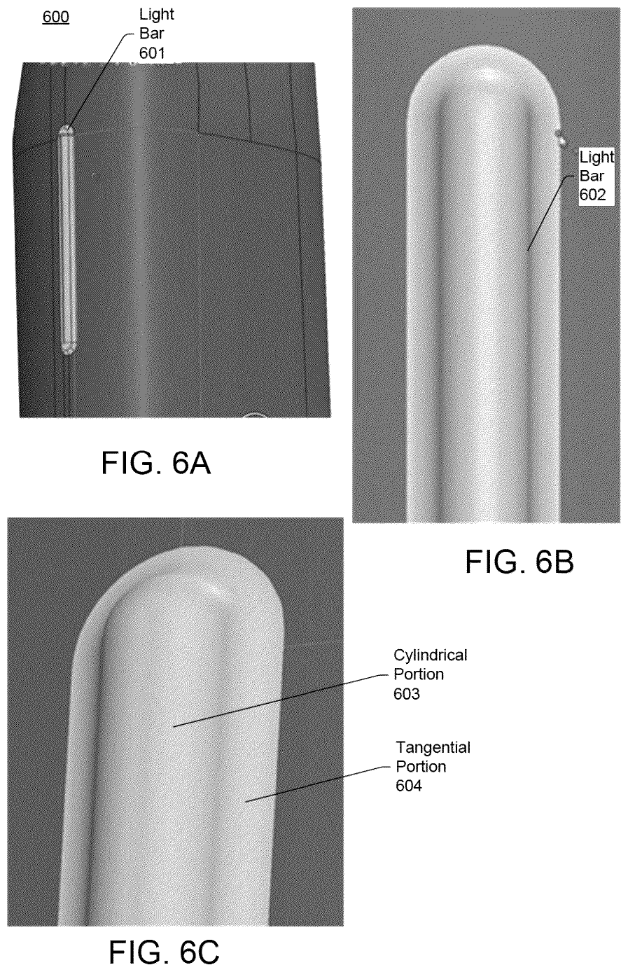

In some embodiments, the scanner 104 includes a light bar that can serve multiple purposes, including to emit light to indicate a status of the scanner or a connection of the scanner, and as a tactile registration marker to indicate by touch an orientation of the scanner 104 when held. These features are described below in more detail with respect to FIGS. 6A-6C.

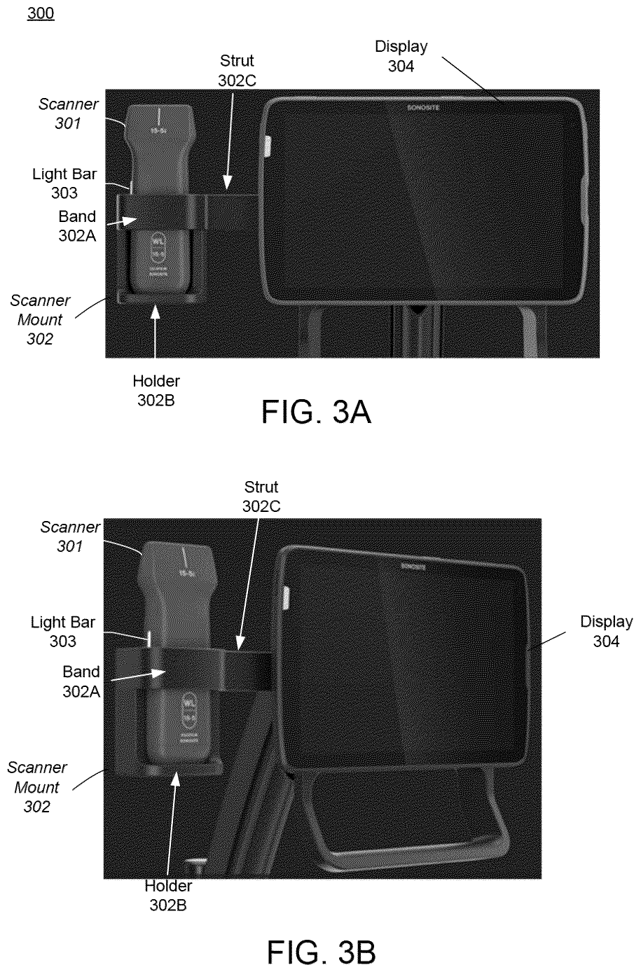

FIGS. 3A and 3B illustrate some embodiments of an ultrasound system 300 including an ultrasound scanner mount attached to a display device mount. Referring to FIGS. 3A and 3B, in some other embodiments, the ultrasound system 300 includes a scanner mount 302 and a display 304.

In some embodiments, the ultrasound system 300 also includes a light bar 303 (or other status indicator) popping out of the top of the scanner mount 301, allowing the user to see the status indicated by the light bar 303 as they approach it. As the user lifts the scanner 301 out of the scanner mount 302, the light bar 303 indicates that the scanner 301 is properly communicably connected (e.g., paired with the ultrasound machine). In some embodiments, while in the scanner mount 302, the light bar 303 on the scanner 301 indicates the charge status of battery; when lifted out, the light bar 303 changes to show the connection status of the scanner 301 to the ultrasound machine 300. In some embodiments, the light bar 303 can be used as an orientation marker to know how to insert the scanner 301 into the scanner mount 302.

In some embodiments, flat area on the scanner 301 is for non-contact charging of a battery (not shown) in the wireless coil under flat surface in scanner 301. In some embodiments a mating charger (coil) is located in the scanner mount 302 between the backing plate (e.g., metal backing plate) (not shown) of the scanner mount 302 and cup (e.g., plastic cup) that holds the scanner 301 as part of the scanner mount 302. Note that in some embodiments arm of the scanner mount 302 is metallic, while other parts are plastic and/or a composite material. In some embodiments, the metal extends all the way down and forms the back of the scanner mount 302. The plastic holder 302B of the scanner mount 302 is for (i) cleanability, and (ii) passing a wireless charging signal (flat spot of the scanner 301 to the back of the holder 302B).

In some embodiments, the scanner mount 302 includes a strut 302C that is configured as an arm or extension for coupling the scanner mount 302 to a display 304 or other part of the ultrasound system 300. In some embodiments, the strut 302C includes a channel to run the cable or other electronic transmission device to a charger that is integrated into the scanner mount 302.

In some embodiments, the scanner 301 can be inserted only one way into the holder. In some embodiments, the open area of holder includes (i) a visual indicator for when the scanner 301 is inserted backwards into the holder 302B, since the open area allows the scanner 301 to be visible from the front of the ultrasound machine 300, and (ii) provides for cleanability on the open area allows access for cleaning the area of the holder 302B where the scanner 301 sits when in the scanner mount 302.

In some embodiments, when the probe is inserted backwards, the scanner 301 still partially fits, so the scanner 301 will not fall off/out of the scanner mount 302. Thus, even if the scanner 301 goes part of the way in the scanner mount 302, the scanner 301 can be retained. In some embodiments, a groove/ledge/retainer allows the scanner 301 to be put part of the way in, but not all the way, thereby preventing the scanner 301 from slipping to the ground and breaking, while visually indicating (by height of the scanner 301) that it is not correctly inserted.

In some embodiments, the scanner mount 302 is a universal holder and is able to hold scanners of many different manufacturers and charge them. In some other embodiments, the scanner mount 302 does not accept probes of certain manufacturers and/or shapes and does not charge such probes.

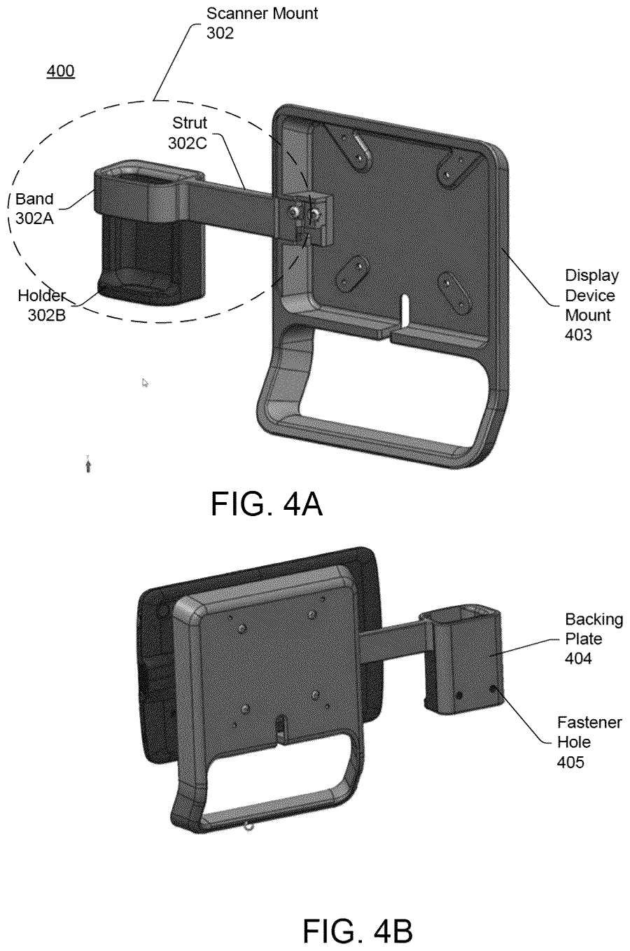

FIGS. 4A and 4B illustrates some embodiments of an ultrasound scanner mount attached to a display device mount. Referring to FIGS. 4A and 4B, the scanner mount 302 is coupled to a display device mount 403 via struct 302C. The display device 403 is configured to hold a display, such as, for example, a display of an ultrasound machine. In some embodiments, the scanner mount 302 includes a back plate 404 that is on or part of the back the scanner mount 302. In some embodiments, the backing plate 404 is metal plate, though it can be made of other materials. In some embodiments, the backing plate 404 includes one or more fastener holes 405. The fastener holes 405 can receive one or more fasteners (e.g., multiple (e.g., 2) screws, etc.). As discussed above, in some embodiments, a charger between the backing plate 404 and the cup of the scanner mount 302 formed with band 302A that holds the scanner 301.

### Example Ultrasound Scanners

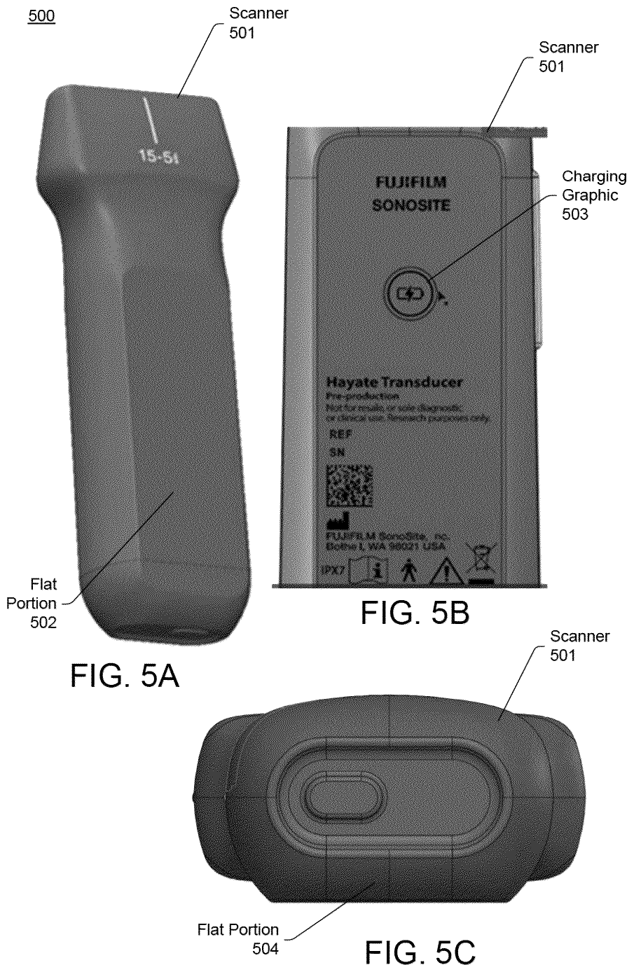

FIGS. 5A-5C illustrate some embodiments of an ultrasound scanner. Referring to FIGS. 5A-5C, the scanner 500 includes a flat portion 502. In some embodiments, the scanner 500 includes a charging indicator, such as, for example, charging graphic 503, that indicates the scanner 500 is charging. In some embodiments, the flat portion 504 is laser marked. Furthermore, in some embodiments, the scanner 500 includes markings on its front-side that indicate one or more of array type, frequency range, connection type (e.g., wired or wireless), etc. In some embodiments, these markings are visible through hole or opening in the scanner mount (e.g., the scanner mount 302).

In some embodiments, the scanner 500 also includes a flat portion 504 that comprises an area for non-contact charging. In some embodiments, a wireless coil (not shown) is under the flat portion 502 in the scanner 500.

FIGS. 6A-6C illustrate some embodiments of light bars for an ultrasound scanner. Referring to FIGS. 6A-6C, an example scanner 600 can include a light bar 601, a light bar 602, or a light bar 603. In some embodiments, the light bar includes a transparent, clear, or frosted material (e.g., plastic, etc.) to cover a light producing element. In some embodiments, the light bar can include one or more light-emitting diodes (LED). In some other embodiments, the light bar includes a laser, halogen, fluorescent, an infra-red (IR) LED illuminator, or any other suitable light emitting element.

In some embodiments, the light bar can be illuminated to provide status information about the scanner via a visual indication to a user of the scanner or any other individual (e.g., a clinician or other medical practitioner). In some embodiments, the light bar is implemented to indicate a status of one parameter when the ultrasound scanner is retained by the band and the holder and another parameter when the ultrasound scanner is not retained by the band and the holder. For example, in some embodiments, the light bar can indicate that the scanner is charging when the ultrasound scanner is retained in the band and the holder and the light bar can indicate a wireless connection status (e.g., the scanner is pairing, whether pairing is successful, etc.) when the ultrasound scanner is not retained by the band and the holder. In some embodiments, the light bar can indicate that the scanner is properly seated in the scanner mount. In this case, the light bar can serve as registration marker on scanner.

The light bar can include a first portion that protrudes from an enclosure of the ultrasound scanner. The first portion can include at least a portion of a cylindrical shape. In some embodiments, the light bar includes a second portion that extends beyond the cylindrical shape and tangentially transitions to the enclosure of the ultrasound scanner. The first portion and the second portion can emit light.

The light bar can output light of a single color or multiple colors. For example, the light bar can display a first color during one mode (e.g., charging) and another color during a second mode (e.g., scanner pairing with the ultrasound system). At times, the light bar can be illuminated with multiple colors at the same time. The light bar can be illuminated with a variety of colors and intensities. The light bar can also be illuminated with repeating or periodic patterns of color and/or intensities. For example, in some embodiments, the light bar can blink.

In some embodiments, the light bar is elongated and disposed along an exterior of one of the scanner (e.g., scanner 301) to provide light to the front of the ultrasound system so that an individual can see the light. In some embodiments, the light bar is disposed on the upper cover of the scanner, attached and/or embedded in the upper surface of the scanner. For example, light bar 601 of FIG. 6A extends down a slide of the scanner at one of its edges, while light bar 602 extends down a cylindrical side of the scanner.

In some embodiment, the light bar extends past the top of the scanner mount enabling it to be visible. In some embodiments, the light bar extends down along the scanner such that it is visible through an opening in the scanner mount. The illumination area of the light bar can extend to the fillet area of the scanner. For example, as shown in FIG. 6C, which is another view of the light bar of FIG. 6B, the illumination does not just occupy are a cylindrical portion 603 of the scanner but also transitions to a tangential portion 604 of the scanner. By doing so, the light from light bar appears to be greater than if the illuminating solely the cylindrical portion 603.

The techniques disclosed herein are not limited to the use of one light bar. In some embodiments, the scanner includes more than one light bar. For example, a scanner can include two light bars. In some embodiments, different light bars are viewable from different sides of the scanner.

### Example Ultrasound Scanner Mounts

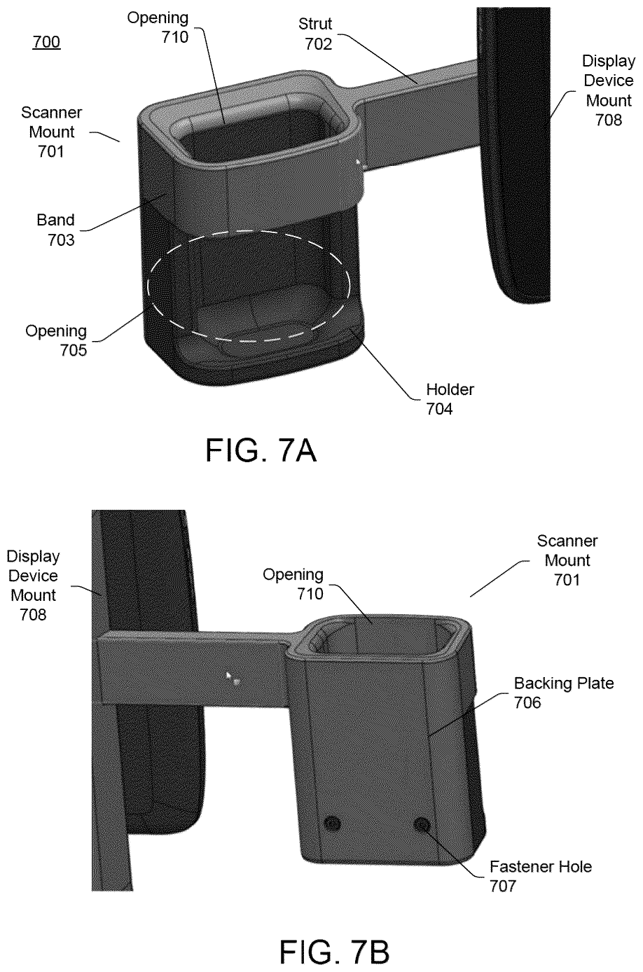

FIGS. 7A and 7B illustrate some embodiments of an ultrasound scanner mount for an ultrasound scanner. Referring to FIGS. 7A and 7B, the scanner mount 701 includes a strut 702 for coupling to a display device mount 705 or some other portion of an ultrasound system. The scanner mount 701 also includes a band 703 that forms an opening 710 at the top of the scanner mount 701 through which a scanner can be inserted and come to rest on holder 704. In some embodiments, there is an opening 704 between the band 703 and the holder 704 through which a portion of the scanner can be viewed. FIG. 7B illustrates the backing plate 706 and the fastener hole 705 on the back of the scanner mount 701. The backing plate 706 can have one or more of the fastener hole 705, with each for receiving a fastener (e.g., a screw, etc.). Such fasteners can be used to secure the holder 704 or some other portion of the scanner mount 701. In some embodiments, the ultrasound scanner receives energy, from a charger via a charger transmitter between the backing plate 706 and the holder 704, that is transferred through the holder 704 when the ultrasound scanner is retained by the band 703 and the holder 704.

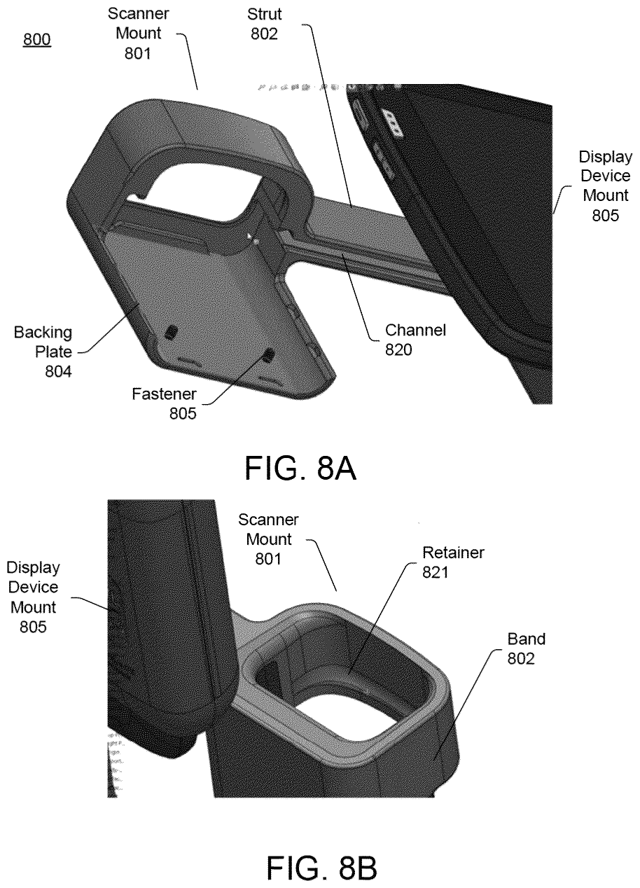

FIGS. 8A and 8B illustrate some other embodiments of an ultrasound scanner mount. Referring to FIGS. 8A and 8B, the scanner mount 801 includes a channel 820 that runs along the strut 802. The channel 820 can be used to retain a cable or other electronic transmission device to a charger that is integrated into the scanner mount 801. For example, the channel 820 can be used to retain a cable coupled to a display device coupled to the display device mount 805 and a charging coil in or attached to the scanner mount 801.

In some embodiments, the band 802 of the ultrasound scanner mount 801 includes a retainer 821. The retainer 821 captures the ultrasound scanner from falling through the band when the ultrasound scanner is inserted incorrectly into the scanner mount. In some embodiments, the band and the holder form an opening though which a portion of the ultrasound scanner is visible when the ultrasound scanner is retained by the band and the holder. When the ultrasound scanner is inserted incorrectly into the scanner mount 801, the retainer 821 can position the ultrasound scanner so that some but not all of the portion of the ultrasound scanner is visible through the opening in the scanner mount 801.

### An Example System

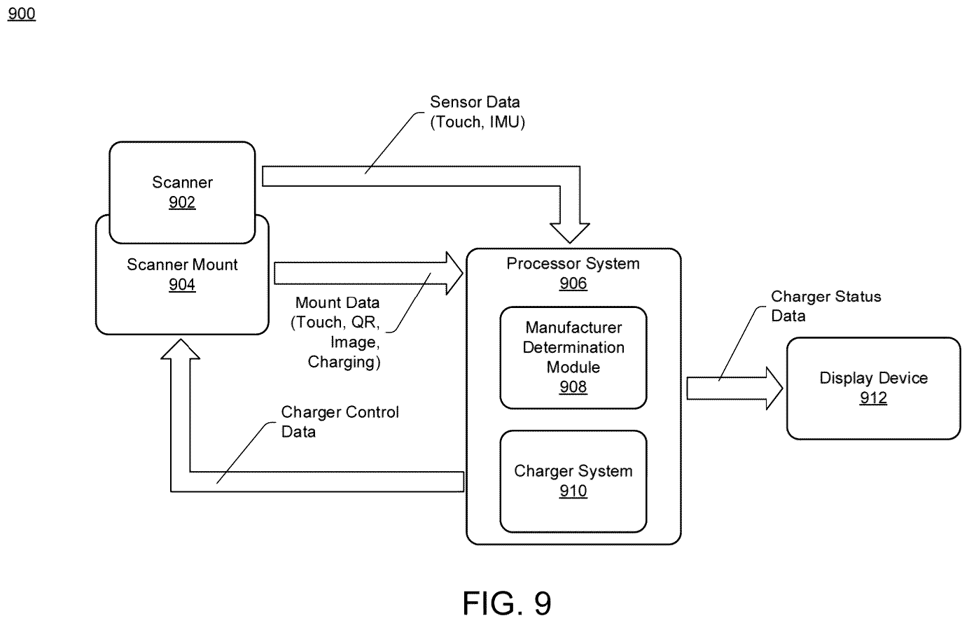

FIG. 9 illustrates an example system 900 in accordance with the some embodiments. In some embodiments, the system 900 is an example of the ultrasound system illustrated in the environment 100 of FIG. 1 and the implementation 200 in FIG. 2. The system 900 includes an ultrasound scanner 902, which is an example of the ultrasound scanner 104 previously described. The system 900 also includes a scanner mount 904, which is an example of the scanner mounts in the previously described figures. The system 900 also includes a processor system 906, which is an example of the processor(s) 106 as previously described. The processor system 906 implements a manufacturer determination module 908 and a charger system 910. The system 900 also includes a display device 912, which is an example of the display device 108 previously described.

The scanner 902 is mounted in the scanner mount 904. The scanner 902 can include touch sensors and/or an IMU, as previously described, and provides touch sensor data and IMU data to the processor system 906. The scanner mount 904 can include touch sensors to determine a position and orientation of the scanner 902 in the scanner mount 904. For instance, the scanner mount 904 can include touch and/or pressure sensors on its internal surfaces. The scanner mount 904 can also include a QR reader implemented to read a QR code on the scanner 902. The scanner mount 904 can also include a camera implemented to generate image data that includes an image of the scanner 902. The scanner mount 904 can also generate charging data indicative of the charging of the scanner 902 based on one or more charging coils implemented in the scanner mount 904 and/or the scanner 902. In some embodiments, the scanner mount 904 provides mount data to the processor system 906. The mount data can include one or more of touch data, QR data, image data, and charging data.

In some embodiments, the processor system 906 receives the sensor data from the scanner 902 and the mount data from the scanner mount 904, and based on one or more of this data, the manufacturer determination module 908 can make a determination of a manufacturer of the scanner 902. In some embodiments, the determination includes an indication of whether the scanner 902 is manufactured by a same manufacturer who manufactures the scanner mount 904. In some embodiments, the manufacturer determination module 908 implements a machine-learned model that processes the sensor data from the scanner 902 and/or the mount data from the scanner mount 904, and generates the determination of whether the scanner 902 and the scanner mount 904 are manufactured by a same manufacturer. In some embodiments, based on the determination and/or the mount data, the charger system 910 generates charger control data to adjust the charging of the scanner 902 while it is mounted in the scanner mount 904. The charger control data can include to adjust the transfer of charging energy to the ultrasound scanner 902 from the scanner mount 904. For example, the adjustment can include to reduce the amount of charging energy transferred to the ultrasound scanner 902. In some embodiments, the adjustment can include to disable the transfer of charging energy to the ultrasound scanner 902.

In some embodiments, the processor system 906 provides charger status data to the display device 912. The charger status data can include any suitable data regarding the charging of the scanner 902 while it is mounted in the scanner mount 904, such as a time remaining to fully charge a battery of the scanner 902, an amount (e.g., percent) of charge of the battery of the scanner 902, and the like. In some embodiments, the charger status data includes a warning indicative of the determination (of the manufacturer of the scanner 902) or the adjustment of the charging, and the display device 912 is implemented to display the warning, such as via text and/or an icon.

### An Example Device

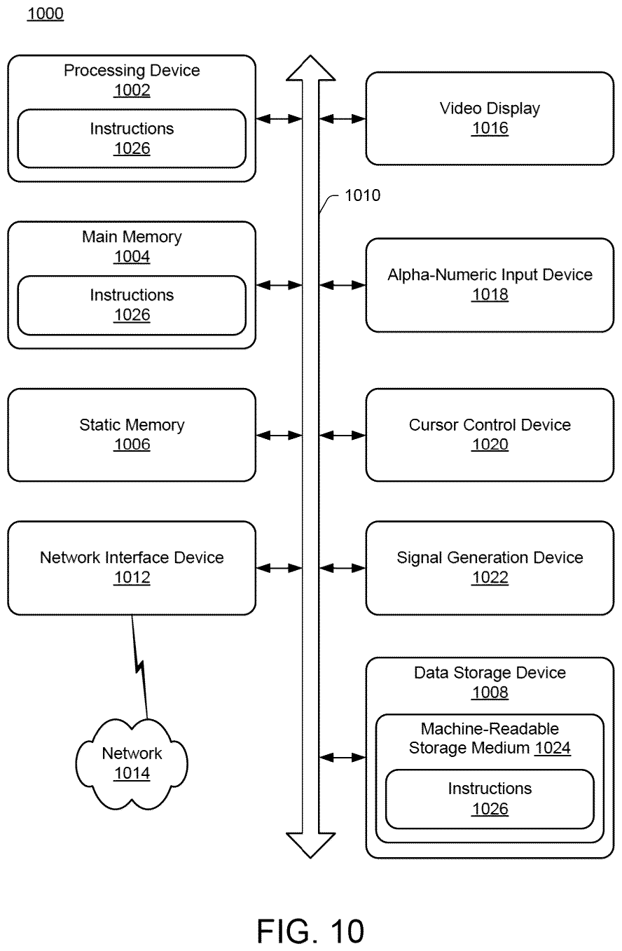

FIG. 10 illustrates a block diagram of some embodiments of computing device 1000 that can perform one or more of the operations described herein, in accordance with some implementations. The computing device 1000 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1000 is one or more of an ultrasound machine, an ultrasound scanner, an access point, a charging station, and a medical archiver.

The computing device 1000 can include a processing device 1002 (e.g., a general-purpose processor, a programmable logic device (PLD), etc.), a main memory 1004 (e.g., synchronous dynamic random-access memory (DRAM), read-only memory (ROM), etc.), and a static memory 1006 (e.g., flash memory, a data storage device 1008, etc.), which can communicate with each other via a bus 1010. The processing device 1002 can be provided by one or more general-purpose processing devices such as a microprocessor, a central processing unit, or the like. In an illustrative example, the processing device 1002 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1002 can also comprise one or more special-purpose processing devices such as an application-specific integrated circuit (ASIC), a field programmable gate array (FPGA), a digital signal processor (DSP), a network processor, or the like. The processing device 1002 can be configured to execute the operations described herein, in accordance with one or more aspects of the present disclosure, for performing the operations and steps discussed herein.

The computing device 1000 can further include a network interface device 1012, which can communicate with a network 1014. The computing device 1000 also can include a video display unit 1016 (e.g., a liquid crystal display (LCD), an organic light-emitting diode (OLED), a cathode ray tube (CRT), etc.), an alphanumeric input device 1018 (e.g., a keyboard), a cursor control device 1020 (e.g., a mouse), and an acoustic signal generation device 1022 (e.g., a speaker, a microphone, etc.). In some embodiments, the video display unit 1016, the alphanumeric input device 1018, and the cursor control device 1020 can be combined into a single component or device (e.g., an LCD touch screen).

The data storage device 1008 can include a computer-readable storage medium 1024 on which can be stored one or more sets of instructions 1026 (e.g., instructions for carrying out the operations described herein, in accordance with one or more aspects of the present disclosure). The instructions 1026 can also reside, completely or at least partially, within the main memory 1004 and/or within the processing device 1002 during execution thereof by the computing device 1000, where the main memory 1004 and the processing device 1002 also constitute computer-readable media. The instructions can further be transmitted or received over the network 1014 via the network interface device 1012.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1008 of the computing device 1000 as executable instructions or code. Although represented as software implementations, the described modules can be implemented as any form of a control application, software application, signal processing and control module, hardware, or firmware installed on the computing device 1000.

While the computer-readable storage medium 1024 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### An Example Environment

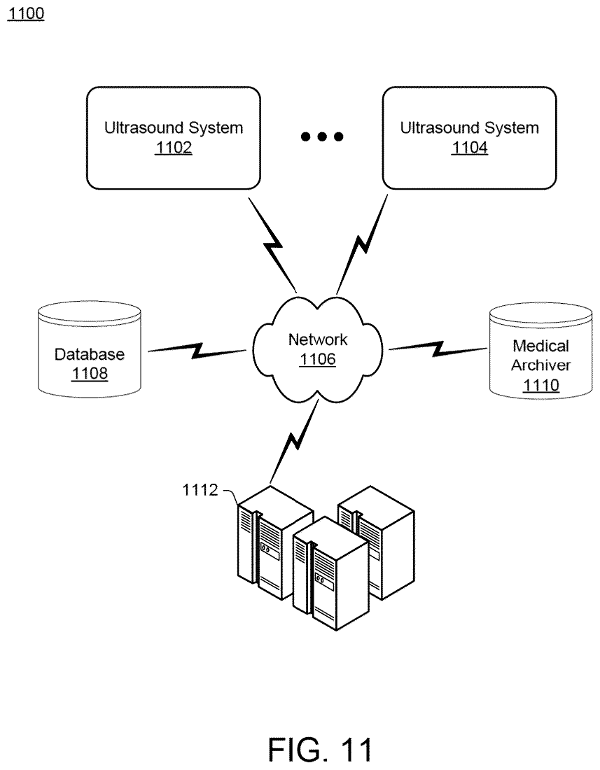

FIG. 11 illustrates some embodiments of an environment 1100 for an ultrasound system. The environment 1100 includes an ultrasound system 1102 and an ultrasound system 1104. Two example ultrasound systems 1102 and 1104 are illustrated in FIG. 11 for clarity. However, the environment 1100 can include any suitable number of ultrasound systems, such as the ultrasound systems maintained by a care facility or the department of a care facility. Generally, an ultrasound system can include any suitable device (e.g., a component of an ultrasound system). In some embodiments, examples devices of the ultrasound systems 1102 and 1104 include a charging station, an ultrasound machine, a display device (e.g., a tablet or smartphone), an ultrasound scanner, and an ultrasound cart. Other examples include a transducer cable, a transducer cable holder, a docking station for an ultrasound machine, a scanner station configured to hold one or more ultrasound scanners, a needle guide, a battery for a wireless ultrasound scanner, a battery for an ultrasound machine, a registration system, a scanner mount, and the like.

The ultrasound systems 1102 and 1104 can be in communication via the network 1106 as part of the environment 1100. The network 1106 can include any suitable network, such as a local area network, a wide area network, a near field communication network, the Internet, an intranet, an extranet, a system bus that couples devices or device components (e.g., in an ASIC, FPGA, or SOC), and combinations thereof. Accordingly, in some embodiments, information can be communicated to the ultrasound systems 1102 and 1104 through the network 1106. For instance, the database 1108 can store instructions executable by a processor system of the ultrasound systems 1102 and 1104, and communicate the instructions via the network 1306.

The environment 1100 can include a medical archiver 1110, e.g., a vendor neutral archive (VNA), that maintains patient medical records. The medical archiver 1110 is coupled to the network 1106, and can be in communication with the ultrasound systems 1102 and 1104 via the network 1106. For instance, the medical archiver 1110 can store medical data (e.g., ultrasound examination data) generated by the ultrasound systems 1102 and 1104, and provide medical data (e.g., data from previous ultrasound examinations) to the ultrasound systems 1102 and 1104 for use in a current ultrasound examination, such as for comparing current and previous waveforms generated during ultrasound examinations.

In some embodiments, the environment 1100 also includes a server system 1112 that can implement any of the functions described herein. The server system 1112 can be a separate device from the ultrasound systems 1102 and 1104. Alternatively, the server system 1112 can be included in at least one of the ultrasound systems 1102 and 1104. In some embodiments, the server system 1112 and the database 1108 are included in at least one of the ultrasound systems 1102 and 1104. In some embodiments, the server system 1112 is implemented as a remote server system that is remote from (e.g., not collocated with) the ultrasound systems 1102 and 1104.

### An Example Machine-Learned Model

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form:

$$f ( \hat{s} , θ ) = \hat{y} \qquad Equation ( 1 )$$

In Equation (1), the operator f represents the processing of the machine-learned model based on an input and providing an output. The term ŝ represents a model input, such as ultrasound data. In some embodiments, the model analyzes/processes the input ŝ using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

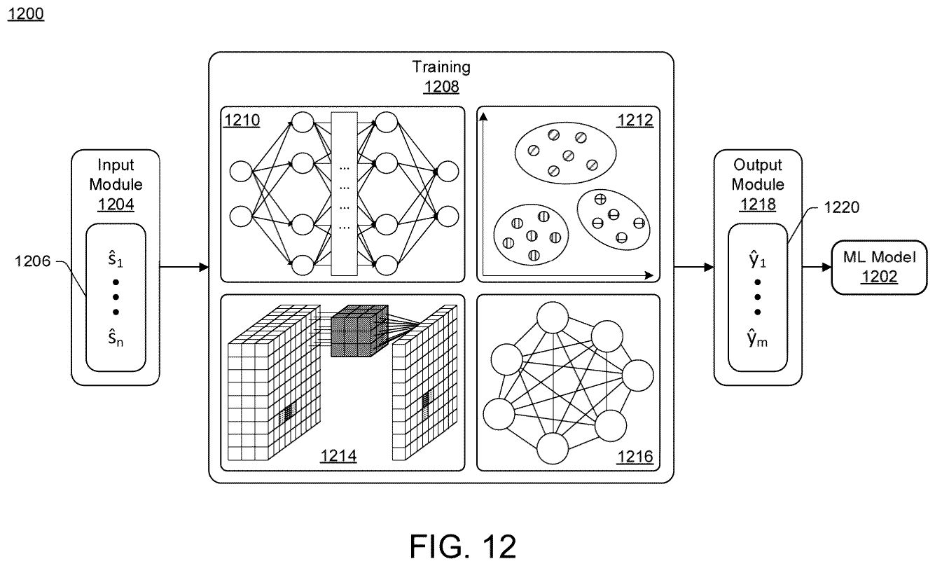

FIG. 12 represents some embodiments of an example machine-learning architecture 1200 used to train a machine-learned model M 1202. An input module 1204 accepts an input s 1206, which can be an array with members ŝ₁ through ŝₙ. The input ŝ 1206 is fed into a training module 1208, which processes the input ŝ 1206 based on the machine-learning architecture 1200. For example, if the machine-learning architecture 1200 uses a multilayer perceptron (MLP) model 1210, the training module 1208 applies weights and biases to the input ŝ 1206 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 1210 is described here as an example, any suitable machine-learning technique can be employed, some examples of which include but are not limited to k-means clustering 1212, convolutional neural networks (CNN) 1214, a Boltzmann machine 1216, Gaussian mixture models (GMM), and long short-term memory (LSTM). The training module 1208 provides an input to an output module 1218. In some embodiments, the output module 1218 analyzes the input from the training module 1208 and provides an output in the form of ŝ 1220, which can be an array with members ŷ₁ through ŷₘ. The output 1220 can represent a known correlation with the input ŝ 1206, such as, for example, object identification, segmentation, and/or classification.

In some embodiments, the input ŝ 1206 can be a training input labeled with known output correlation values, and these known values can be used to optimize the output ŷ 1220 in training against the optimization/loss function. In some other embodiments, the machine-learning architecture 1200 can categorize the output ŝ 1220 values without being given known correlation values to the inputs ŝ 1206. In some embodiments, the machine-learning architecture 1200 can be a combination of machine-learning architectures. By way of example, a first network can use the input ŝ 1206 and provide the output ŝ 1220 as an input SML to a second machine-learned architecture, with the second machine-learned architecture providing a final output ŷ_{f}. In some other embodiments, one or more machine-learning architectures can be implemented at various points throughout the training module 1208.

In some machine-learned models, all layers of the model are fully connected. For example, all perceptrons in an MLP model act on every member of s. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

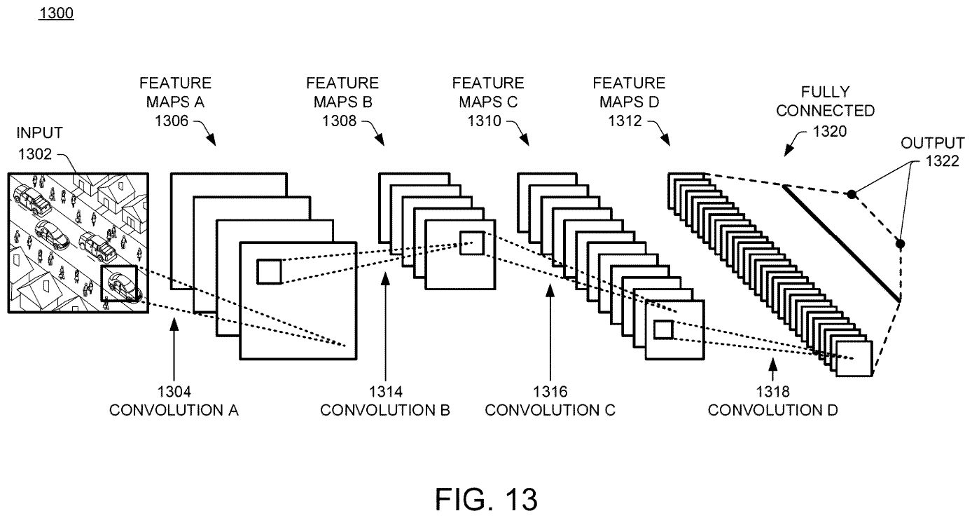

FIG. 13 represents some embodiments an example model 1300 using a CNN to process an input image 1302, which includes representations of objects that can be identified via object recognition, such as people or cars (or an anatomy, as described in relation to FIGS. 1-12). Convolution A 1304 can be performed to create a first set of feature maps (e.g., feature maps A 1306). A feature map can be a mapping of aspects of the input image 1302 given by a filter element of the CNN. This process can be repeated using feature maps A 1306 to generate further feature maps B 1308, feature maps C 1310, and feature maps D 1312 using convolution B 1314, convolution C 1316, and convolution D 1318, respectively. In some embodiments, the feature maps D 1312 become an input for fully connected network layers 1320. In this way, the machine-learned model can be trained to recognize certain elements of the image, such as people, cars, or a particular patient anatomy, and provide an output 1322 that, for example, identifies the recognized elements. In some embodiments, an inference generated with an ultrasound system can be appended to a feature map (e.g., feature map B 1308) generated by a neural network (e.g., CNN). In this way, the feature vector and/or inference can be used as a secondary/conditional input to the neural network.

Although the example of FIG. 13 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art can also be employed.

### Example Procedures

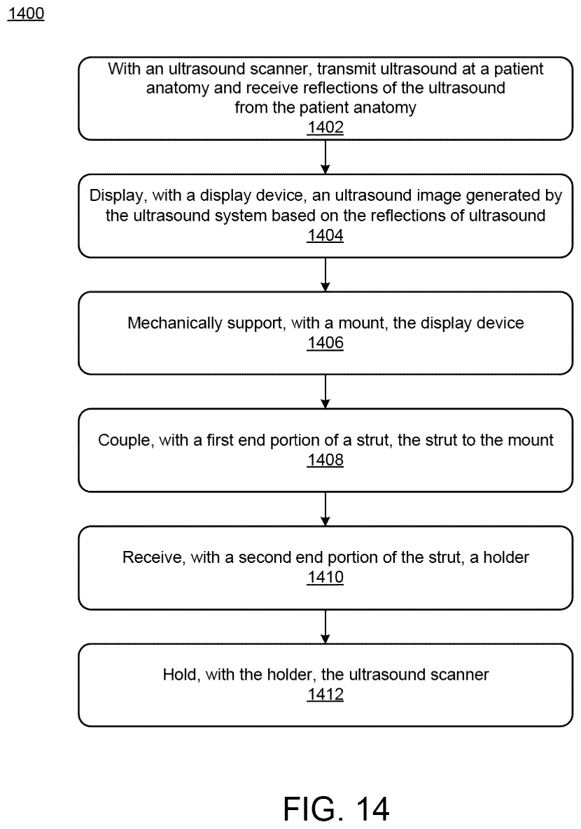

FIG. 14 illustrates some embodiments of an example method 1400 that can be implemented by an ultrasound system (e.g., the ultrasound system of FIG. 1) for determining venous congestion with ultrasound. The ultrasound system can include an ultrasound scanner (e.g., transducer or probe), an ultrasound machine, a scanner mount, a processor system, a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof.

With an ultrasound scanner, ultrasound is transmitted at a patient anatomy and reflections of the ultrasound from the patient anatomy are received (block 1402). With a display device, an ultrasound image generated by the ultrasound system based on the reflections of ultrasound is displayed (block 1404). The display device is mechanically supported with a mount (block 1406). A strut is coupled to the mount with a first end portion of the strut (block 1408). A holder is received with a second end portion of the strut (block 1410). The ultrasound scanner is held with the holder (block 1412).

In some embodiments, the second end portion of the strut includes a backing plate implemented to secure the holder to the strut. The ultrasound system can include a first charging coil positioned between the backing plate and the holder. The first charging coil can provide a charging energy through the holder to a second charging coil included in the ultrasound scanner. In some embodiments, the strut includes a channel implemented to retain a cable coupled to the first charging coil and the display device. In some embodiments, the strut includes a first material and the holder includes a second material that is different from the first material.

In some embodiments, the second charging coil is implemented under a flat portion of an enclosure of the ultrasound scanner. The flat portion of the enclosure can be implemented to register insertion of the ultrasound scanner into a band included in the second end portion of the strut. The band can surround a perimeter of the ultrasound scanner when the ultrasound scanner is held by the holder. The flat portion of the enclosure can include a graphic indicating to charge the ultrasound scanner at the flat portion of the enclosure.

In some embodiments, the second end portion of the strut includes a band implemented to surround a perimeter of the ultrasound scanner so that when the ultrasound scanner is held by the holder, the second end portion of the strut and the holder form an opening through which markings on the ultrasound scanner are visible. The markings can indicate at least one of a model of the ultrasound scanner, a frequency range of the ultrasound scanner, a type of array included in the ultrasound scanner, and a type of connection between the ultrasound scanner and the display device. In some embodiments, the opening provides cleaning access to the holder when the ultrasound scanner is removed from the holder.

In some embodiments, the second end portion of the strut includes a band implemented to surround a perimeter of the ultrasound scanner. In some embodiments, the band includes a retainer that, when the ultrasound scanner is inserted backwards into the band, allows the ultrasound scanner to be partially and not wholly inserted, and prevents the ultrasound scanner from falling through the band.

In some embodiments, the ultrasound scanner includes a light. The second end portion of the strut can include a band implemented to surround a perimeter of the ultrasound scanner so that when the ultrasound scanner is held by the holder, the light is at least partially viewable. In an example, when the ultrasound scanner is held by the holder, the light indicates a battery status of a battery of the ultrasound scanner. When the ultrasound scanner is removed from the holder, the light can change from indicating the battery status to indicating a connection status (e.g., paired or not paired, etc.) of a wireless connection between the ultrasound scanner and the display device. In some embodiments, the light is implemented as a light bar that includes a first portion that protrudes from an enclosure of the ultrasound scanner. The first portion of the light bar that protrudes can indicate an orientation of the ultrasound scanner. Additionally or alternatively, the light bar can include a second portion that tangentially transitions to the enclosure of the ultrasound scanner.

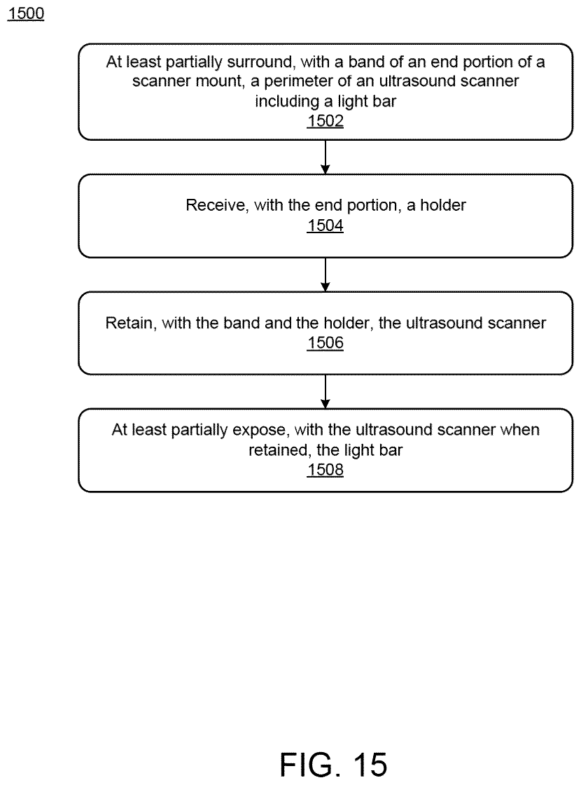

FIG. 15 illustrates some other embodiments of an example method 1500 that can be implemented by an ultrasound system (e.g., the ultrasound system of FIG. 1) for determining venous congestion with ultrasound. The ultrasound system can include an ultrasound scanner (e.g., transducer or probe), an ultrasound machine, a scanner mount, a processor system, a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof.

In some embodiments, a perimeter of an ultrasound scanner including a light bar is at least partially surrounded with a band of an end portion of a scanner mount (block 1502). A holder is received with the end portion (block 1504). The ultrasound scanner is retained with the band and the holder (block 1506). The light bar is at least partially exposed when the ultrasound scanner is retained (block 1508).

In some embodiments, the light bar is implemented to indicate a status of a first parameter when the ultrasound scanner is retained by the band and the holder and a status of a second parameter when the ultrasound scanner is not retained by the band and the holder. The light bar can include a first portion that protrudes from an enclosure of the ultrasound scanner. The first portion can include at least a portion of a cylindrical shape. In some embodiments, the light bar includes a second portion that extends beyond the cylindrical shape and tangentially transitions to the enclosure of the ultrasound scanner. The first portion and the second portion can emit light.

In some embodiments, the ultrasound system includes a charger transmitter positioned between the end portion and the holder. The charger transmitter can transfer a charging energy to the ultrasound scanner when the ultrasound scanner is retained by the band and the holder. In some embodiments, the end portion is comprised of a material that does not pass the charging energy and the holder is comprised of an additional material that does pass the charging energy. For example, the material can be a metal and the additional material can be a plastic.

In some embodiments, the band includes a retainer that captures the ultrasound scanner from falling through the band when the ultrasound scanner is inserted incorrectly into the scanner mount. In some embodiments, the band and the holder form an opening though which a portion of the ultrasound scanner is visible when the ultrasound scanner is retained by the band and the holder. The retainer, when the ultrasound scanner is inserted incorrectly into the scanner mount, can position the ultrasound scanner so that some but not all of the portion of the ultrasound scanner is visible through the opening.

In some embodiments, the end portion includes a fastener hole and is implemented to receive the holder via a fastener inserted through the fastener hole. The fastener can include one or more fasteners, such as two screws.

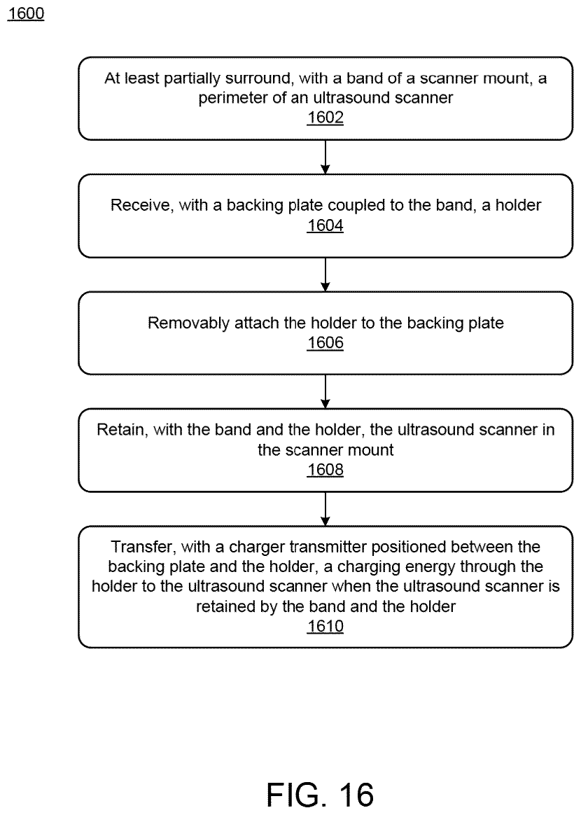

FIG. 16 illustrates some embodiments of an example method 1600 that can be implemented by an ultrasound system (e.g., the ultrasound system of FIG. 1) for determining venous congestion with ultrasound. The ultrasound system can include an ultrasound scanner (e.g., transducer or probe), an ultrasound machine, a scanner mount, a processor system, a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof.

A perimeter of an ultrasound scanner is at least partially surround with a band of a scanner mount (block 1602). A holder is received by a backing plate coupled to the band (block 1604). A holder is removably attached to the backing plate (block 1606). The ultrasound scanner in the scanner mount is retained with the band and the holder (block 1608). A charging energy is transferred through the holder to the ultrasound scanner when the ultrasound scanner is retained by the band and the holder (block 1610). The charger energy can be transferred with a charger transmitter positioned between the backing plate and the holder.

In some embodiments, the backing plate is coupled to the band by a manufacturing process of a monolithic material that comprises the backing plate and the band. The manufacturing process can include at least one of a casting, a milling, or a three dimensional printing of the monolithic material to form the backing plate and the band as a single piece. In some embodiments, the backing plate is coupled to the band by removably attaching the backing plate to the band.

In some embodiments, the band includes a retainer that captures the ultrasound scanner from falling through the band when the ultrasound scanner is inserted incorrectly into the scanner mount. In some embodiments, the band and the holder can form an opening though which a portion of the ultrasound scanner is visible when the ultrasound scanner is retained by the band and the holder. The retainer, when the ultrasound scanner is inserted incorrectly into the scanner mount, can position the ultrasound scanner so that some but not all of the portion of the ultrasound scanner is visible through the opening. In some embodiments, the portion of the ultrasound scanner includes markings that indicate at least one of a model of the ultrasound scanner, a frequency range of the ultrasound scanner, a type of array included in the ultrasound scanner, and a type of connection between the ultrasound scanner and an ultrasound machine. In some embodiments, some of the portion does not include at least some of the markings.

In some embodiments, the scanner mount includes a processor system implemented to determine that the ultrasound scanner retained in the scanner mount is not manufactured by a manufacturer of the scanner mount. In some embodiments, the processor system can adjust, responsive to the determination, the transfer of the charging energy to the ultrasound scanner. In some embodiments, the adjustment includes to reduce the amount of charging energy transferred to the ultrasound scanner. Additionally or alternatively, the adjustment can include to disable the transfer of charging energy to the ultrasound scanner. In some embodiments, the determination is based on the transfer of the charging energy to the ultrasound scanner. For instance, the processor system can monitor the amount, rate, and/or profile of the charging energy coupled to the ultrasound scanner to make the determination.

In some embodiments, the scanner mount includes touch sensors implemented to generate sensor data indicating contact points between the ultrasound scanner and the scanner mount when the ultrasound scanner is retained by the band and the holder. The determination can be based on the sensor data. Additionally or alternatively, the scanner mount can include a quick response (QR) reader implemented to read a QR code on the scanner, and the determination can be based on the reading of the QR code. In some embodiments, the processor system is coupled to a display device that is implemented to display a warning indicative of the determination and/or the adjustment.

There are a number of example embodiments described herein.

Example 1 is an ultrasound system including an ultrasound scanner configured to transmit ultrasound at a patient anatomy and receive reflections of the ultrasound from the patient anatomy, a display device configured to display an ultrasound image generated by the ultrasound system based on the reflections of ultrasound, and a mount configured to mechanically support the display device. The ultrasound system also includes a strut including a first end portion and a second end portion, where the first end portion is configured to couple the strut to the mount, the second end portion configured to receive a holder, and the holder is configured to hold the ultrasound scanner.

Example 2 is the ultrasound system of example 1 that may optionally include that the second end portion of the strut includes a backing plate implemented to secure the holder to the strut.

Example 3 is the ultrasound system of example 2 that may optionally include that the ultrasound system includes a first charging coil positioned between the backing plate and the holder, the first charging coil implemented to provide a charging energy through the holder to a second charging coil included in the ultrasound scanner.

Example 4 is the ultrasound system of example 3 that may optionally include that the strut includes a channel implemented to retain a cable coupled to the first charging coil and the display device.

Example 5 is the ultrasound system of example 3 that may optionally include that the strut includes a first material and the holder includes a second material that is different from the first material.

Example 6 is the ultrasound system of example 3 that may optionally include that the second charging coil is implemented under a flat portion of an enclosure of the ultrasound scanner, where the flat portion of the enclosure is implemented to register insertion of the ultrasound scanner into a band included in the second end portion of the strut.

Example 7 is the ultrasound system of example 6 that may optionally include that the band is implemented to surround a perimeter of the ultrasound scanner when the ultrasound scanner is held by the holder.

Example 8 is the ultrasound system of example 6 that may optionally include that the flat portion of the enclosure includes a graphic indicating to charge the ultrasound scanner at the flat portion of the enclosure.

Example 9 is the ultrasound system of example 1 that may optionally include that the second end portion of the strut includes a band implemented to surround a perimeter of the ultrasound scanner so that when the ultrasound scanner is held by the holder, where the second end portion of the strut and the holder form an opening through which markings on the ultrasound scanner are visible.

Example 10 is the ultrasound system of example 9 that may optionally include that the markings indicate at least one of a model of the ultrasound scanner, a frequency range of the ultrasound scanner, a type of array included in the ultrasound scanner, and a type of connection between the ultrasound scanner and the display device.

Example 11 is the ultrasound system of example 9 that may optionally include that the opening provides cleaning access to the holder when the ultrasound scanner is removed from the holder.

Example 12 is the ultrasound system of example 1 that may optionally include that the second end portion of the strut includes a band implemented to surround a perimeter of the ultrasound scanner, the band including a retainer that, when the ultrasound scanner is inserted backwards into the band, allows the ultrasound scanner to be partially and not wholly inserted, and prevents the ultrasound scanner from falling through the band.

Example 13 is the ultrasound system of example 1 that may optionally include that the ultrasound scanner includes a light, wherein the second end portion of the strut includes a band implemented to surround a perimeter of the ultrasound scanner so that when the ultrasound scanner is held by the holder, the light is at least partially viewable.

Example 14 is the ultrasound system of example 13 that may optionally include that when the ultrasound scanner is held by the holder, the light indicates a battery status of a battery of the ultrasound scanner.

Example 15 is the ultrasound system of example 14 that may optionally include that when the ultrasound scanner is removed from the holder, the light changes from indicating the battery status to indicating a connection status of a wireless connection between the ultrasound scanner and the display device.

Example 16 is the ultrasound system of example 13 that may optionally include that the light is implemented as a light bar that includes a first portion that protrudes from an enclosure of the ultrasound scanner.

Example 17 is the ultrasound system of example 16 that may optionally include that the first portion of the light bar that protrudes indicates an orientation of the ultrasound scanner.

Example 18 is the ultrasound system of example 16 that may optionally include that light bar includes a second portion that tangentially transitions to the enclosure of the ultrasound scanner.

Example 19 is an ultrasound system including an ultrasound scanner, which includes a light bar, and a scanner mount including an end portion having a band configured to at least partially surround a perimeter of the ultrasound scanner, where the end portion is configured to receive a holder, the band and the holder is configured to retain the ultrasound scanner, and the ultrasound scanner when retained is configured to at least partially expose the light bar.

Example 20 is the ultrasound system of example 19 that may optionally include that the light bar is implemented to indicate a status of a first parameter when the ultrasound scanner is retained by the band and the holder and a status of a second parameter when the ultrasound scanner is not retained by the band and the holder.

Example 21 is the ultrasound system of example 19 that may optionally include that the light bar includes a first portion that protrudes from an enclosure of the ultrasound scanner.

Example 22 is the ultrasound system of example 21 that may optionally include that the first portion includes at least a portion of a cylindrical shape.

Example 23 is the ultrasound system of example 22 that may optionally include that light bar includes a second portion that extends beyond the cylindrical shape and tangentially transitions to the enclosure of the ultrasound scanner, wherein the first portion and the second portion emit light.

Example 24 is the ultrasound system of example 19 that may optionally include a charger transmitter positioned between the end portion and the holder, and implemented to transfer a charging energy to the ultrasound scanner when the ultrasound scanner is retained by the band and the holder.

Example 25 is the ultrasound system of example 24 that may optionally include that the end portion is comprised of a material that does not pass the charging energy and the holder is comprised of an additional material that does pass the charging energy.

Example 26 is the ultrasound system of example 25 that may optionally include that the material is a metal and the additional material is a plastic.

Example 27 is the ultrasound system of example 19 that may optionally include that the band includes a retainer that captures the ultrasound scanner from falling through the band when the ultrasound scanner is inserted incorrectly into the scanner mount.

Example 28 is the ultrasound system of example 27 that may optionally include that the band and the holder form an opening though which a portion of the ultrasound scanner is visible when the ultrasound scanner is retained by the band and the holder, and wherein the retainer, when the ultrasound scanner is inserted incorrectly into the scanner mount, positions the ultrasound scanner so that some but not all of the portion of the ultrasound scanner is visible through the opening.

Example 29 is the ultrasound system of example 19 that may optionally include that the end portion includes a fastener hole and is implemented to receive the holder via a fastener inserted through the fastener hole.

Example 30 is a scanner mount for holding an ultrasound scanner, where the scanner mount includes a band configured to at least partially surround a perimeter of the ultrasound scanner, and a backing plate coupled to the band and configured to receive a holder, where the holder is configured to be removably attached to the backing plate, and the band and the holder are configured to retain the ultrasound scanner in the scanner mount. The scanner mount also includes a charger transmitter positioned between the backing plate and the holder and configured to transfer a charging energy through the holder to the ultrasound scanner when the ultrasound scanner is retained by the band and the holder.

Example 31 is the scanner mount of example 30 that may optionally include that the backing plate is coupled to the band by a manufacturing process of a monolithic material that comprises the backing plate and the band.

Example 32 is the scanner mount of example 30 that may optionally include that the manufacturing process includes at least one of a casting, a milling, or a three dimensional printing of the monolithic material to form the backing plate and the band as a single piece.

Example 33 is the scanner mount of example 30 that may optionally include that the backing plate is coupled to the band by removably attaching the backing plate to the band.

Example 34 is the scanner mount of example 30 that may optionally include that the band includes a retainer that captures the ultrasound scanner from falling through the band when the ultrasound scanner is inserted incorrectly into the scanner mount.

Example 35 is the scanner mount of example 34 that may optionally include that the band and the holder form an opening though which a portion of the ultrasound scanner is visible when the ultrasound scanner is retained by the band and the holder, and wherein the retainer, when the ultrasound scanner is inserted incorrectly into the scanner mount, positions the ultrasound scanner so that some but not all of the portion of the ultrasound scanner is visible through the opening.

Example 36 is the scanner mount of example 35 that may optionally include that the portion of the ultrasound scanner includes markings that indicate at least one of a model of the ultrasound scanner, a frequency range of the ultrasound scanner, a type of array included in the ultrasound scanner, and a type of connection between the ultrasound scanner and an ultrasound machine, and wherein said some of the portion does not include at least some of the markings.

Example 37 is the scanner mount of example 30 that may optionally include a processor system implemented to: determine that the ultrasound scanner retained in the scanner mount is not manufactured by a manufacturer of the scanner mount; and adjust, responsive to the determination, the transfer of the charging energy to the ultrasound scanner.

Example 38 is the scanner mount of example 37 that may optionally include that the adjustment includes to reduce the amount of charging energy transferred to the ultrasound scanner.

Example 39 is the scanner mount of example 37 that may optionally include that the adjustment includes to disable the transfer of charging energy to the ultrasound scanner.

Example 40 is the scanner mount of example 37 that may optionally include that the determination is based on the transfer of the charging energy to the ultrasound scanner.

Example 41 is the scanner mount of example 37 that may optionally include that the scanner mount includes touch sensors implemented to generate sensor data indicating contact points between the ultrasound scanner and the scanner mount when the ultrasound scanner is retained by the band and the holder, and wherein the determination is based on the sensor data.

Example 42 is the scanner mount of example 37 that may optionally include that the scanner mount includes a quick response (QR) reader implemented to read a QR code on the scanner, and wherein the determination is based on the reading of the QR code.

Example 43 is the scanner mount of example 37 that may optionally include that the processor system is coupled to a display device that is implemented to display a warning indicative of the determination or the adjustment.

Example 40 is an ultrasound system that performs the operations of one or more of examples 1-29.

Example 41 is a method including the operations performed by the ultrasound system of one or more of examples 1-29.

Example 42 is a method including the operations performed by the scanner mount of one or more of examples 30-43.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. An ultrasound system comprising:
an ultrasound scanner configured to transmit ultrasound at a patient anatomy and receive reflections of the ultrasound from the patient anatomy;
a display device configured to display an ultrasound image generated by the ultrasound system based on the reflections of ultrasound;
a mount configured to mechanically support the display device;
a strut including a first end portion and a second end portion, the first end portion configured to couple the strut to the mount, the second end portion configured to receive a holder; and
the holder configured to hold the ultrasound scanner.

2. The ultrasound system as described in claim 1, wherein the second end portion of the strut includes a backing plate implemented to secure the holder to the strut.

3. The ultrasound system as described in claim 2, wherein the ultrasound system includes a first charging coil positioned between the backing plate and the holder, the first charging coil implemented to provide a charging energy through the holder to a second charging coil included in the ultrasound scanner.

4. The ultrasound system as described in claim 3, wherein the strut includes a channel implemented to retain a cable coupled to the first charging coil and the display device.

5. The ultrasound system as described in claim 3, wherein the strut includes a first material and the holder includes a second material that is different from the first material.

6. The ultrasound system as described in claim 3, wherein the second charging coil is implemented under a flat portion of an enclosure of the ultrasound scanner, the flat portion of the enclosure implemented to register insertion of the ultrasound scanner into a band included in the second end portion of the strut.

7. The ultrasound system as described in claim 6, wherein the band is implemented to surround a perimeter of the ultrasound scanner when the ultrasound scanner is held by the holder.

8. The ultrasound system as described in claim 1, wherein the second end portion of the strut includes a band implemented to surround a perimeter of the ultrasound scanner so that when the ultrasound scanner is held by the holder, the second end portion of the strut and the holder form an opening through which markings on the ultrasound scanner are visible.

9. The ultrasound system as described in claim 8, wherein the markings indicate at least one of a model of the ultrasound scanner, a frequency range of the ultrasound scanner, a type of array included in the ultrasound scanner, and a type of connection between the ultrasound scanner and the display device.

10. The ultrasound system as described in claim 8, wherein the opening provides cleaning access to the holder when the ultrasound scanner is removed from the holder.

11. (canceled)

12. (canceled)

13. (canceled)

14. (canceled)

15. (canceled)

16. (canceled)

17. (canceled)

18. (canceled)

19. (canceled)

20. (canceled)

21. The ultrasound system as described in claim 6, wherein the flat portion of the enclosure includes a graphic indicating to charge the ultrasound scanner at the flat portion of the enclosure.

22. A scanner mount for holding an ultrasound scanner, comprising:
a band configured to at least partially surround a perimeter of the ultrasound scanner;
a backing plate coupled to the band and configured to receive a holder;
the holder configured to be removably attached to the backing plate,
wherein the band and the holder form an opening through which a portion of the ultrasound scanner is visible when the ultrasound scanner is retained by the band and the holder; and
a charger transmitter positioned between the backing plate and the holder and configured to transfer charging energy through the holder to the ultrasound scanner when the ultrasound scanner is retained by the band and the holder.

23. The scanner mount as described in claim 22, wherein the backing plate is coupled to the band by a manufacturing process of a monolithic material that comprises the backing plate and the band.

24. The scanner mount as described in claim 22, wherein the manufacturing process includes at least one of a casting, a milling, or a three dimensional printing of the monolithic material to form the backing plate and the band as a single piece.

25. The scanner mount as described in claim 22, wherein the backing plate is coupled to the band by removably attaching the backing plate to the band.

26. The scanner mount as described in claim 22, wherein the band includes a retainer that captures the ultrasound scanner from falling through the band when the ultrasound scanner is inserted incorrectly into the scanner mount.

27. The scanner mount as described in claim 26, wherein the retainer, when the ultrasound scanner is inserted incorrectly into the scanner mount, positions the ultrasound scanner so that some but not all of the portion of the ultrasound scanner is visible through the opening.

28. The scanner mount as described in claim 27, wherein the portion of the ultrasound scanner includes markings that indicate at least one of a model of the ultrasound scanner, a frequency range of the ultrasound scanner, a type of array included in the ultrasound scanner, and a type of connection between the ultrasound scanner and an ultrasound machine, and wherein said some of the portion does not include at least some of the markings.

29. The scanner mount as described in claim 22, further comprising a processor system implemented to:
determine that the ultrasound scanner retained in the scanner mount is not manufactured by a manufacturer of the scanner mount; and
adjust, responsive to the determination, the transfer of the charging energy to the ultrasound scanner.

30. The scanner mount as described in claim 29, wherein the adjustment includes to reduce the amount of charging energy transferred to the ultrasound scanner or includes to disable the transfer of charging energy to the ultrasound scanner.
